(19) United States
(12) Patent Application Publication
(10) Pub. No.: US 2005/0025829 A1
(43) Pub. Date: Feb. 3, 2005

(54) Controlled drug release tablets

(76) Inventor: Cherng-Ju Kim, Highland, CA (US)

Correspondence Address: SHELDON & MAK, INC, 225 SOUTH LAKE AVENUE, 9TH FLOOR, PASADENA, CA, 91101, US

(21) Appl. No.: 10/630,879

(22) Filed: Jul. 29, 2003

(51) Int. Cl. A61K 9/24

(52) U.S. Cl. 424/471

(57) ABSTRACT

A pharmaceutical composition in the form of a perforated tablet comprising one or more than one enteric polymer and a drug, where the enteric polymer is substantially hydrophobic and substantially soluble in a substantially aqueous environment above a pH of about 5. The pharmaceutical composition can additionally comprise one or more than one layer, and can additionally comprise one or more than one binder.

### BACKGROUND

Orally administering drugs to patients, including humans, over extended time and at a controlled release rate is advantageous in some medical situations. It is also advantageous in some medical situations for the release rate of a drug to follow zero-order kinetics, meaning that the drug is released at a constant linear release rate, or a substantially constant linear release rate over time. It is also advantageous to release a drug in the intestine and not in the stomach to protect the stomach from the harmful effects caused by many drugs and to protect the drugs from the effects of gastric fluid. It is also advantageous for a pharmaceutical composition to have a tablet form that is amenable to large scale manufacturing processes.

Extended release pharmaceutical compositions are currently known. Examples of extended release pharmaceutical compositions include monolithic matrices, geometrically modified tablets, membrane reservoirs, swellable polymers, dissolvable polymers, ion exchange resins, and osmosis. Disadvantageously, however, these pharmaceutical compositions do not also exhibit zero-order release kinetics or are not produced in a form that is amenable to large scale manufacturing processes. For example, monolithic matrices, fabricated from water-insoluble polymers, a drug and excipients, exhibit first-order release kinetics or square-root-of-time kinetics, meaning that the drug release rate decreases over time. Geometrically modified tablets, such as semi-hemispheric, pie-shaped and multi-holed shaped tablets, that are surface coated with water-insoluble polymers and impermeable polymers are not practically produced in large scale manufacturing processes.

Controlled release pharmaceutical compositions are also currently known. Disadvantageously, however, these controlled release pharmaceutical compositions do not exhibit a suitably extended release time, or the controlled release of the drug does not follow zero-order kinetics. For example, perforated coated tablets, formed with a central hole and using water-soluble excipients, exhibit a constant or slightly increasing drug release rate over time. However, the drug is released from these tablets in an unsuitably short three hour time period. Perforated coated tablets, formed with a central hole and using a water-insoluble polymer as a drug carrier are also known. Disadvantageously, however, the drug release rate and time is dependent on the amount of water-insoluble polymer that is used in these tablets. For example, if the content of the water-insoluble polymer in the tablet is high, square-root-of-time kinetics with a prolonged release time, such as greater than 20 hours, is observed. Whereas, if the content of the water-insoluble polymer in the tablet is low, a constant release rate with a short ten hour release time is observed.

A controlled release, hydrophilic polymer based pharmaceutical composition in a donut-shaped tablet form is also known. This pharmaceutical composition is formed from a mixture of a hydrophilic polymer, a drug, and excipients. Disadvantageously, however, the drug release rate of this pharmaceutical composition is dependent on the solubility of the drug and drug loading. For example, the release rate of poorly water-soluble drugs from the donut-shaped tablet follows zero-order kinetics and the release rate of highly water-soluble drugs from the donut-shaped tablet follows anomalous kinetics. Another problem associated with hydrophilic polymer based pharmaceutical compositions in a donut-shaped tablet form is that these tablets can dose dump, that is, when not fully hydrated the hydrophilic polymers become very viscous and adhere to solids and biological surfaces. The surface of the tablet then peels off and the drug dosage is dumped into the patient.

Pharmaceutical compositions based on erodible and swellable/erodible polymers with a central hole in a coated donut-shaped tablet form are also known. The drug release rate of these pharmaceutical compositions follows parabolic and zero-order release kinetics. Disadvantageously, however, drug release is significantly slowed or may stop once viscous liquids or foods are placed in the central hole.

Therefore, there is a need for a pharmaceutical composition in a tablet form that has a zero-order, or a substantially zero order kinetic drug release rate over extended time, where the pharmaceutical composition does not adhere to solids and biological surfaces, thereby leading to dose dumping, and the drug release is not stopped by physical interaction of the tablet with other elements, such as food. It would also be desirable for the pharmaceutical composition to release the drug in the intestine and for the tablet form to be amenable to large scale manufacturing processes.

### SUMMARY

According to the present invention, there is provided a pharmaceutical composition for the controlled release of a drug in the form of a perforated tablet. In one embodiment, the pharmaceutical composition comprises one or more than one enteric polymer and a drug, where the enteric polymer is substantially hydrophobic and substantially soluble in a substantially aqueous environment above a pH of about 5. In one preferred embodiment, the pharmaceutical composition comprises a plurality of layers, where one or more than one of the plurality of layers is a substantially water-insoluble polymer, or a substantially water-soluble polymer, and where one or more than one of the plurality of layers comprises an enteric polymer and a drug. In another preferred embodiment, the form of the perforated tablet is a cylindrically shaped tablet, where the perforation extends completely through the center of the cylindrically shaped tablet. In another preferred embodiment, the one or more than one enteric polymer is selected from the group consisting of a hydroxypropylmethylcellulose acetate succinate, a hydroxypropylmethylcellulose phthalate, a polyvinylacetate, and a polyacrylate. Preferably, the one or more than one enteric polymer is a polyacrylate selected from the group consisting of an acrylate polymer, a methacrylate polymer, a methylmethacrylate polymer, an ethylacrylate polymer, a copolymer comprising a combination of the preceding polymers, and a carboxylic acid functional group containing derivative of the preceding polymers and copolymers. More preferably, the one or more than one enteric polymer is a polyacrylate selected from the group consisting of a methacrylic acid-methylmethacrylate copolymer and a methacrylic acid-ethylacrylate copolymer. In another preferred embodiment, the one or more than one enteric polymer is present in an amount effective to control the release of the drug at a substantially constant linear rate over time, or a slightly increasing linear rate over time, or a slightly decreasing linear rate over time. Preferably, the enteric polymer is present in an amount of between about 1% and about 99%. More preferably, the enteric polymer is present in an amount of between about 20% and about 75%, and most preferably, the enteric polymer is present in an amount of between about 35% and about 65%. In another preferred embodiment, the pharmaceutical composition additionally comprises one or more than one binder. Preferably, the binder is selected from the group consisting of a water-soluble cellulose, a polyethylene oxide, a polyethylene glycol, a water-insoluble cellulose, a water-insoluble polyvinylacetate, and a water-insoluble polyacrylate. More preferably, the one or more than one binder is a water-insoluble polyacrylate selected from the group consisting of a water-insoluble acrylate polymer, a water-insoluble methacrylate polymer, a water-insoluble methylmethacrylate polymer, a water-insoluble ethylacrylate polymer, a copolymer comprising a combination of the preceding polymers, a water-insoluble quaternary ammonium functional group containing derivative of the preceding polymers and copolymers, and a water-insoluble ester functional group containing derivative of the preceding polymers and copolymers. Most preferably, the one or more than one binder is a water-insoluble polyacrylate selected from the group consisting of an acrylate-methacrylate copolymer with a quaternary ammonium functional group, an ethylacrylate-methylmethacrylate copolymer with a neutral ester functional group, and an (ethylacrylate, methylmethacrylate) polymer dispersion.

In another embodiment according to the present invention, there is provided a method of making a pharmaceutical composition for the controlled release of a drug in the form of a perforated tablet. In one embodiment, the method comprises: a) mixing one or more than one enteric polymer and a drug to form a mixture, where the enteric polymer is substantially hydrophobic and substantially soluble in a substantially aqueous environment above a pH of about 5; b) compressing the mixture into a tablet; and c) forming a perforation in the tablet. In a preferred embodiment, the method additionally comprises mixing one or more than one binder into the one or more than one enteric polymer and the drug to form the mixture.

In another embodiment according to the present invention, there is provided a pharmaceutical composition for the controlled release of a drug in the form of a perforated tablet. In one embodiment, the pharmaceutical composition comprises: a) one or more than one outer layer comprising one or more than one substantially water-insoluble polymer, or one or more than one substantially water-soluble polymer; and b) an inner layer comprising one or more than one enteric polymer and a drug; where the enteric polymer is substantially hydrophobic and substantially soluble in an aqueous environment above a pH of about 5. In one preferred embodiment, the form of the perforated tablet is a cylindrically shaped tablet, where the perforation extends completely through the center of the cylindrically shaped tablet. In another preferred embodiment, the one or more than one enteric polymer is selected from the group consisting of a hydroxypropylmethylcellulose acetate succinate, a hydroxypropylmethylcellulose phthalate, a polyvinylacetate, and a polyacrylate. Preferably, the one or more than one enteric polymer is a polyacrylate selected from the group consisting of an acrylate polymer, a methacrylate polymer, a methylmethacrylate polymer, an ethylacrylate polymer, a copolymer comprising a combination of the preceding polymers, and carboxylic acid functional group containing derivative of the preceding polymers and copolymers. More preferably, the one or more than one enteric polymer is a polyacrylate selected from the group consisting of a methacrylic acid-methylmethacrylate copolymer and a methacrylic acid-ethylacrylate copolymer. In another preferred embodiment, the one or more than one enteric polymer is present in an amount effective to control the release of the drug at a substantially constant linear rate over time, or a slightly increasing linear rate over time, or a slightly decreasing linear rate over time. Preferably, the enteric polymer is present in an amount of between about 1% and about 99%. More preferably, the enteric polymer is present in an amount of between about 20% and about 75%, and most preferably, the enteric polymer is present in an amount of between about 35% and about 65%. In another preferred embodiment, the inner layer further comprises one or more than one binder. Preferably, the binder is selected from the group consisting of a water-soluble cellulose, a polyethylene oxide, a polyethylene glycol, a water-insoluble cellulose, a water-insoluble polyvinylacetate, and a water-insoluble polyacrylate. More preferably, the one or more than one binder is a water-insoluble polyacrylate selected from the group consisting of a water-insoluble acrylate polymer, a water-insoluble methacrylate polymer, a water-insoluble methylmethacrylate polymer, a water-insoluble ethylacrylate polymer, a copolymer comprising a combination of the preceding polymers, a water-insoluble quaternary ammonium functional group containing derivative of the preceding polymers and copolymers, and a water-insoluble ester functional group containing derivative of the preceding polymers and copolymers, and most preferably, the one or more than one binder is a water-insoluble polyacrylate selected from the group consisting of an acrylate-methacrylate copolymer with a quaternary ammonium functional group, an ethylacrylate-methylmethacrylate copolymer with a neutral ester functional group, and an (ethylacrylate, methylmethacrylate) polymer dispersion. In another preferred embodiment, the outer layer comprises one or more than one substantially water-insoluble polymer selected from the group consisting of a water-insoluble ethylcellulose, a water-insoluble cellulose ester, a water-insoluble polyvinylacetate, and a water-insoluble polyacrylate. Preferably, the outer layer comprises one or more than one water-insoluble polyacrylate selected from the group consisting of a water-insoluble acrylate polymer, a water-insoluble methacrylate polymer, a water-insoluble methylmethacrylate polymer, a water-insoluble ethylacrylate polymer, a copolymer comprising a combination of the preceding polymers, a water-insoluble quaternary ammonium functional group containing derivative of the preceding polymers and copolymers, and a water-insoluble ester functional group containing derivative of the preceding polymers and copolymers. More preferably, the outer layer comprises one or more than one water-insoluble polyacrylate selected from the group consisting of an acrylate-methacrylate copolymer with a quaternary ammonium functional group, an ethylacrylate-methylmethacrylate copolymer with a neutral ester functional group, and an (ethylacrylate, methylmethacrylate) polymer dispersion. In another preferred embodiment, the outer layer comprises one or more than one substantially water-soluble polymer selected from the group consisting of a water-soluble cellulose, a water-soluble polyethylene oxide, and a polysaccharide.

In another embodiment according to the present invention, there is provided a method of making a pharmaceutical composition for the controlled release of a drug in the form of a perforated tablet, where the pharmaceutical composition comprises one or more than one outer layer comprising one or more than one substantially water-insoluble polymer, or one or more than one substantially water-soluble polymer; and an inner layer comprising one or more than one enteric polymer and a drug, where the enteric polymer is hydrophobic and substantially soluble in an aqueous environment above a pH of about 5. In one embodiment the method comprises: a) compressing a first outer layer into a die; b) mixing an inner layer comprising one or more than one enteric polymer and a drug to form an inner layer mixture; c) compressing the inner layer mixture into the first outer layer; d) compressing a second outer layer into the inner layer mixture to form a tablet; and e) forming a perforation in the tablet. In one preferred embodiment, the method additionally comprises mixing one or more than one binder into the mixture comprising the one or more than one enteric polymer and a drug to form the mixture.

### FIGURES

These and other features, aspects and advantages of the present invention will become better understood from the following description, appended claims, and accompanying figures where:

FIG. 1 is a top side perspective view of a perforated tablet according to one embodiment of the present invention;

FIG. 2 is a top side perspective view of a perforated layered tablet according to another embodiment of the present invention;

FIG. 3 is a superimposed graph of the kinetic drug release rate in water and the kinetic drug release rate in a pH 6.8 solution of glipizide from a perforated tablet according to another embodiment of the present invention;

FIG. 4 is a superimposed graph of the kinetic drug release rate of glipizide from a perforated tablet according to another embodiment of the present invention and the kinetic drug release rate of a brand name form of glipizide, Glucotrol® XL;

FIG. 5 is a superimposed graph of the kinetic drug release rate of nifedipine from a perforated tablet according to another embodiment of the present invention and the kinetic drug release rates of 30 mg, 60 mg, and 90 mg dosage forms of a brand name form of nifedipine, Procardia® XL; and

FIG. 6 is a graph of the kinetic drug release rate of glipizide from a perforated layered tablet according to another embodiment of the present invention.

### DESCRIPTION

According to one embodiment of the present invention, there is provided a pharmaceutical composition for the controlled release of a drug. The pharmaceutical composition comprises a perforated tablet and one or more than one enteric polymer and a drug. The enteric polymer is substantially hydrophobic and substantially soluble at enteric pH, that is, a pH of above about 5, and substantially insoluble at low pH, that is a pH below about 5. Because the pharmaceutical composition dissolves at enteric pH, when consumed, the pharmaceutical composition will pass through the stomach without substantially releasing the drug until the pharmaceutical composition reaches the intestine. Further, because the enteric polymers are also hydrophobic and do not adhere to solid and biological surfaces, such as the gastrointestinal tract, the pharmaceutical composition does not dose dump.

According to another embodiment of the present invention, the pharmaceutical composition comprises a perforated tablet that is a cylindrically shaped tablet with a perforation extending completely through the center of the tablet. Using an enteric polymer in a perforated tablet form enhances the drug release kinetics. The pharmaceutical composition releases a drug dosage over an extended time and the kinetic drug release rate is at a constant, or substantially constant linear release rate, thereby following a zero-order kinetic rate. Further, even if the pharmaceutical composition is plugged in the central perforation with food or viscous liquids, drug release is not stopped.

As used in this disclosure, the term “controlled drug release rate” means a drug release rate that is moderated over time.

As used in this disclosure, the term “drug” means the active form of a substance intended for use in the diagnosis, cure, pallation, treatment or prevention of disease.

As used in this disclosure, the term “drug release” means the discharge of the active form of a drug from a carrier.

As used in this disclosure, the term “enteric pH” means a pH above about 5 and below about 8.

As used in this disclosure, the term “extended drug release” means a drug release that is prolonged over time.

As used in this disclosure, the term “water-insoluble” means insolubility of a substance at about 1 mg/L or less at neutral pH.

As used in this disclosure, the term “water-soluble” means a solubility of a substance at about 1 mg/L or higher at neutral pH.

As used in this disclosure, the term “comprise” and variations of the term, such as “comprising” and “comprises,” are not intended to exclude other additives, components, integers or steps.

All amounts disclosed herein are given in weight percent of the total weight of the composition.

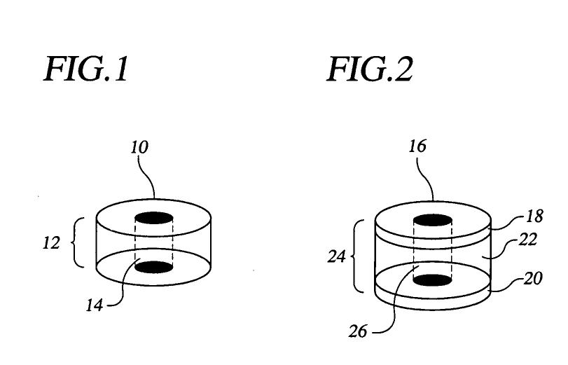

In one embodiment, the present invention is a pharmaceutical composition comprising a perforated tablet for the controlled release of a drug. Referring to FIG. 1, there is shown a top perspective view of a perforated tablet 10 according to the present invention comprising one or more than one enteric polymer and a drug, where the enteric polymer is hydrophobic and substantially soluble in an aqueous environment above a pH of about 5. In another embodiment, the perforated tablet 10 further comprises one or more than one binder. In a preferred embodiment, the perforated tablet 10 comprises a cylindrically shaped tablet 12 with a perforation 14 that extends completely through the center of the tablet. However, the perforated tablet of the present invention can comprise other shapes and tablet perforation configurations, as will be understood by those of skill in the art with reference to this disclosure.

In another embodiment, the present invention is a perforated layered tablet for the controlled release of a drug. Referring to FIG. 2, there is shown a top perspective view of a perforated layered tablet 16 according to the present invention, comprising one or more than one outer polymer layer 18 and 20, where the outer polymer layer 18 and 20 comprises one or more than one substantially water-insoluble polymer, or the outer polymer layer 18 and 20 comprises one or more than one substantially water-soluble polymer, and an inner polymer layer 22 comprising one or more than one enteric polymer and a drug, where the enteric polymer is hydrophobic and substantially soluble in an aqueous environment above a pH of about 5. In one embodiment, the inner polymer layer 22 further comprises one or more than one binder. In a preferred embodiment, the perforated layered tablet 16, comprises a cylindrically shaped tablet 24 with a perforation 26 that extends completely through the center of the layered tablet 16. Other shapes and tablet perforation configurations can be used in the perforated layered tablet of the present invention, as will be understood by those of skill in the art with reference to this disclosure.

In one embodiment, the pharmaceutical composition comprises a hydrophobic enteric polymer that is substantially soluble at enteric pH, that is, the enteric polymer dissolves in a substantially aqueous environment at about 1 mg/L or higher at room temperature. In a preferred embodiment, the enteric polymer is selected from the group consisting of a hydroxypropylmethylcellulose acetate succinate, a hydroxypropylmethylcellulose phthalate, a polyvinylacetate, and a polyacrylate such as an acrylate polymer, a methacrylate polymer, a methylmethacrylate polymer, an ethylacrylate polymer, a copolymer comprising a combination of the preceding polymers, and a carboxylic acid functional group containing polycrylate derivative of the preceding polymers and copolymers. In a more preferred embodiment, the one or more than one binder is selected from the group consisting of a methacrylic acid-methylmethacrylate copolymer, such as Eudragit® and Eudragit® available from Rohm America, LLC., Piscataway, N.J. US; and a methacrylic acid-ethylacrylate copolymer, such as Kollicoat® MAE available from BASF Corp., Mount Olive, N.J. US. Other enteric polymers can be used in the pharmaceutical composition of the present invention, as will be understood by those of skill in the art with reference to this disclosure.

In another embodiment, the pharmaceutical composition additionally comprises one or more than one binder. In a preferred embodiment, the one or more than one binder is selected from the group consisting of a water-soluble cellulose, a polyethylene oxide, a polyethylene glycol, a water-insoluble cellulose, a water-insoluble polyvinylacetate, and a water-insoluble polyacrylate such as a water-insoluble acrylate polymer, a water-insoluble methacrylate polymer, a water-insoluble methylmethacrylate polymer, a water-insoluble ethylacrylate polymer, a copolymer comprising a combination of a preceding water-insoluble polymers, a water-insoluble quaternary ammonium functional group containing derivative of the preceding polymers and copolymers, and a water-insoluble ester functional group containing derivative of the preceding polymers and copolymers. In a more preferred embodiment, the one or more than one binder is a water-insoluble polyacrylate selected from the group consisting of an acrylate-methacrylate copolymer with a quaternary ammonium functional group and an ethylacrylate-methylmethacrylate copolymer with a neutral ester functional group, such as Eudragit® NE and Eudragit® RL, available from Rohm America, LLC., Piscataway, N.J. US; and an (ethylacrylate, methylmethacrylate) polymer dispersions such as Kollicoat® EMM, available from BASF Corp., Mount Olive, N.J. US. Other binders can be used in the pharmaceutical composition of the present invention, as will be understood by those of skill in the art with reference to this disclosure.

In another embodiment of the invention, the outer polymer layer comprises one or more than one water-insoluble polymer layer. In a preferred embodiment, the one or more than one water-insoluble polymer layer is selected from the group consisting of a water-insoluble cellulose, such as an ethylcellulose and a cellulose ester, a water-insoluble polyvinylacetate such as Kollicoat® SR 30D, available from BASF Corp., Mount Olive, N.J. US; and Sentry Plus® polyvinylacetate resin, available from Dow Chemical Co., Midland, Mich. US., and a water-insoluble polyacrylate such as a water-insoluble acrylate polymer, a water-insoluble methacrylate polymer, a water-insoluble methylmethacrylate polymer, and a water-insoluble ethylacrylate polymer, a copolymer comprising a combination of a preceding water-insoluble polyacrylate, a quaternary ammonium functional group containing derivative of the preceding polymers and copolymers, and a neutral ester functional group containing derivative of the preceding polymers and copolymers. In a more preferred embodiment, the one or more than one water-insoluble polyacrylate is selected from the group consisting of an acrylate-methacrylate copolymer with a quaternary ammonium functional group and an ethylacrylate-methylmethacrylate copolymer with a neutral ester functional group, such as Eudragit® NE and Eudragit® RL, available from Rohm America, LLC., Piscataway, N.J. US; and an (ethylacrylate, methylmethacrylate) polymer dispersion such as Kollicoat® EMM, available from BASF Corp., Mount Olive, N.J. US. Other water-insoluble polymers can be used in the outer polymer layer of present invention, as will be understood by those of skill in the art with reference to this disclosure.

In another embodiment, the outer polymer layer comprises one or more than one water-soluble polymer layer. In a preferred embodiment, the one or more than one water-soluble polymer layer is selected from the group consisting of a water-soluble cellulose, a water-soluble polyethylene oxide such as POLYOX®, available from Dow Chemical Co., Midland, Mich. US. Other water-soluble polymers can be used in the outer polymer layer of the present invention, as will be understood by those of skill in the art with reference to this disclosure.

In another preferred embodiment, the weight percent of one enteric polymer, or a combination of more than one enteric polymer in the pharmaceutical composition comprises an amount of between about 1% to about 99% of the total weight percent of the pharmaceutical composition. The kinetic drug release rate from the pharmaceutical composition follows a zero-order kinetic drug release rate, or near zero-order kinetic drug release rate, that is, a substantially constant linear rate over time, or a slightly increasing linear rate over time, or a slightly decreasing linear rate over time. In a more preferred embodiment, the pharmaceutical composition comprises one or more than one enteric polymer in an amount effective to control the release of the drug at a substantially constant linear rate over time, or a slightly increasing linear rate over time, or a slightly decreasing linear rate over time. In a more preferred embodiment, one enteric polymer, or a combination of more than one enteric polymer in the pharmaceutical composition comprises an amount of between about 20% and about 75% of the total weight percent of the pharmaceutical composition. In a most preferred embodiment, one enteric polymer, or a combination of more than one enteric polymer in the pharmaceutical composition comprises an amount of between about 35% and about 65% of the total weight percent of the pharmaceutical composition. However, as will be understood by those of skill in the art with reference to this disclosure, the weight percent of an enteric polymer or combination of enteric polymers used in a particular pharmaceutical composition will vary depending on the particular drug and the enteric polymer combination of enteric polymers used in a particular formulation and other weight percentages can be used according to the present invention.

In another embodiment, the present invention is a method of making a pharmaceutical composition in the form of a perforated tablet comprising one or more than one enteric polymer and a drug, where the enteric polymer is hydrophobic and substantially soluble in an aqueous environment above a pH of about 5. In one embodiment, the method comprises making a pharmaceutical composition in the form of a perforated tablet, such as the perforated tablet 10 shown in FIG. 1. The method comprises providing a pharmaceutical composition in the form of a perforated tablet, where the pharmaceutical composition comprises one or more than one enteric polymer and a drug, and where the enteric polymer is substantially hydrophobic and substantially soluble in an aqueous environment above a pH of about 5. Then, the one or more than one enteric polymer and the drug are combined to form a mixture. Next, the mixture is compressed into a tablet. Next, a perforation is formed in the tablet. In a preferred embodiment, the tablet is a cylindrically shaped tablet and the perforation extends completely through the center of the cylindrically shaped tablet. In another embodiment, the method additionally comprises mixing one or more than one binder into the mixture comprising the enteric polymer and the drug and compressing the binder, the enteric polymer, and the drug into a tablet. In a most preferred embodiment, the method comprises compressing the mixture into a tablet with a press. Next, a drill forms a perforation in the tablet. In one embodiment, the press is a Carver press and the tablet is compressed under about a 6,000 psi compression force. Other methods for fabricating perforated tablets of the present invention can be used, such as modifying punches of a rotary tablet press, as used, for example in a commercial one-step process, as will be understood by those of skill in the art with reference to this disclosure.

In another embodiment, the present invention is a method of making a pharmaceutical composition in the form of a perforated layered tablet. In one embodiment, the method comprises making pharmaceutical composition in the form of a perforated layered tablet, such as the perforated layered tablet 20 shown in FIG. 2. The method comprises providing a pharmaceutical composition comprising one or more than one first outer layer 22, where the outer layer comprises one or more than one substantially water-insoluble polymer, or the outer layer comprises one or more than one substantially water-soluble polymer; and an inner polymer layer comprising one or more than one enteric polymer and a drug, where the enteric polymer is substantially hydrophobic and substantially soluble in a substantially aqueous environment above a pH of about 5. Then, the first outer polymer layer 22 is compressed into a die. Next, the enteric polymer and a drug are combined to form a mixture. Next, the mixture is compressed into the first outer polymer layer 22 to form an inner polymer layer 26. Next, a second outer polymer layer 24 is compressed into the inner polymer layer 26 to form a tablet 28. Next, a perforation 30 is formed in the tablet 28. In a preferred embodiment, the tablet is a cylindrically shaped tablet and the perforation extends completely through the center of the cylindrically shaped tablet. In another embodiment, the method additionally comprises mixing one or more than one binder into the enteric polymer and the drug to form the mixture and compressing the mixture into the first outer polymer layer 22. In a more preferred embodiment, the method comprises compressing the first outer polymer layer 22 into a die with a press. Then, the inner polymer layer 26 is compressed into the first outer polymer layer 22 in the die. Next, the second outer polymer layer 24 is compressed into the inner polymer layer 26 in the die to form the layered tablet 28. Next, the perforation 30 is formed in the layered tablet 28 with a drill. In one embodiment, the press is Carver press and the layered tablet is compressed under about a 6,000 psi compression force. Other methods for fabricating perforated layered tablets of the present invention can be used, such as modifying punches of a rotary multilayer tablet press, as used, for example in a commercial process, as will be understood by those of skill in the art with reference to this disclosure.

### EXAMPLE I

### Kinetic Drug Release Rate of a Perforated Tablet

According to one embodiment of the present invention, a composition for a perforated tablet comprises the substances listed in Table I.

TABLE I

| | PERCENT WEIGHT |
|---|---|
| SUBSTANCE | OF TOTAL WEIGHT |
| Ethylcellulose | 19.34 |
| Eudragit ® S | 19.34 |
| Kollicoat ® MAE | 19.34 |
| Hydroxypropylmethylcellulose acetate succinate | 19.34 |
| Methylcellulose E15 | 19.34 |
| Glipizide | 3.3 |

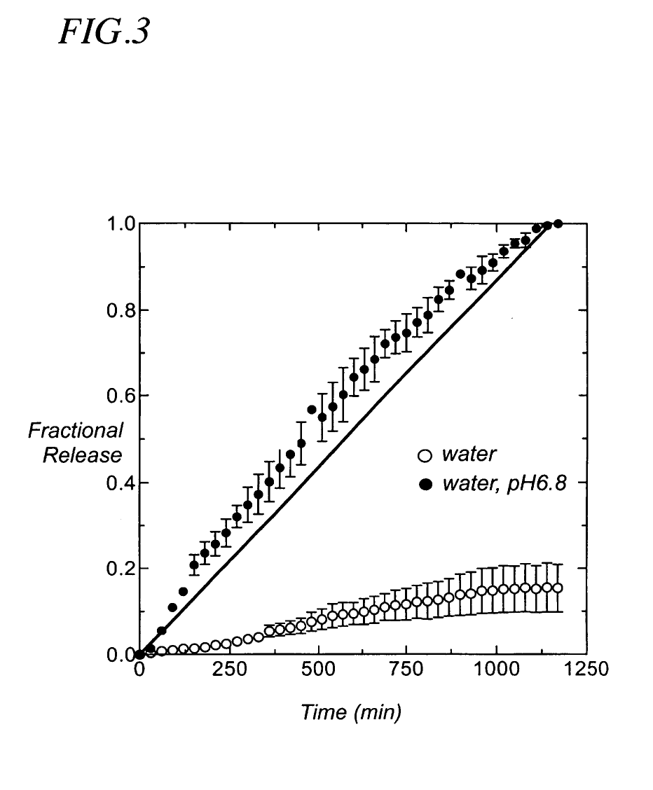

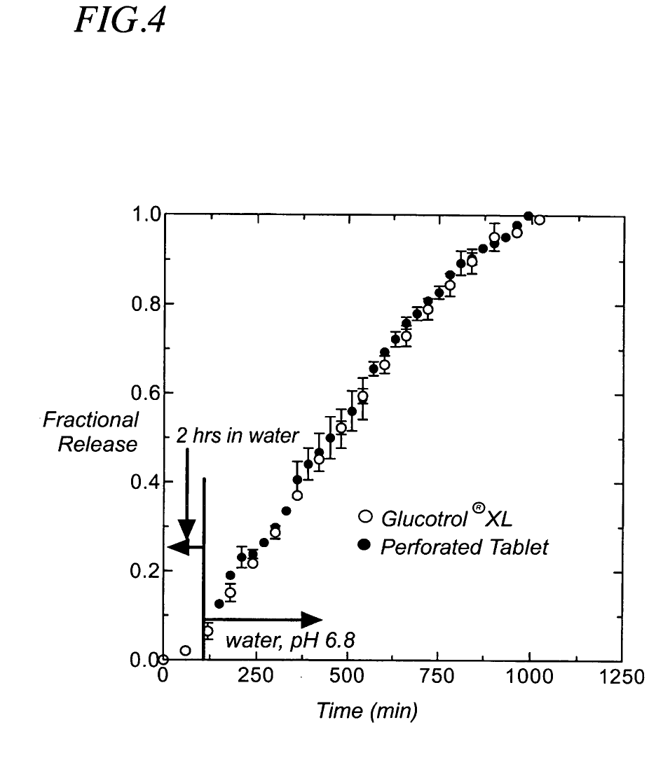

Referring to FIG. 3, there is shown a graph of the kinetic drug release rate of a pharmaceutical composition comprising a perforated tablet and the substances listed in Table I. FIG. 3 shows the kinetic drug release rate in water and the kinetic drug release rate in a pH 6.8 solution prepared from 0.01 M NaH₂PO₄ and 0.01 M Na₂HPO₄ in 0.1 M NaCl, at 50 rpm, of a pharmaceutical composition comprising the substances listed in Table I in a perforated tablet form comprising a 300 mg, 9 mm diameter tablet with a 3.2 mm perforation. As shown in FIG. 3, the kinetic drug release rate of glipizide in water was substantially slowed as compared to the kinetic drug release rate of glipizide in the pH 6.8 solution. As also shown in FIG. 3, the kinetic drug release rate of glipizide from the perforated tablet is near zero-order, that is, a substantially constant linear release rate. Theoretically, the variation from zero-order kinetics is due to a decrease in the lateral surface area of the perforated tablet over time, while the radial surface area of the perforated tablet is maintained over time. A theoretical kinetic drug release rate can be expressed by:

$$\frac{{M}_{t}}{{M}_{∞}} = 2 ( \frac{1}{{1}_{o}} + \frac{1}{{r}_{o} - {r}_{i}} - \frac{2 {k}_{e} t}{{C}_{o} {1}_{o} ( {r}_{o} - {r}_{i} )} ) t \qquad ( 1 )$$

where Mₜ/M_{∞} is the fractional release, kₑ is the constant erosion rate of enteric polymers, rₒ is the radius of the tablet, rᵢ is the radius of the central hole, lₒ is the thickness of the tablet, Cₒ is the initial drug concentration in the tablet, and t is the release time.

### EXAMPLE II

### Comparative Kinetic Drug Release Rates

In another embodiment of the present invention, the kinetic drug release rate of a pharmaceutical composition in the form of a perforated tablet and the substances listed in Table I was compared to the kinetic drug release rate of a pharmaceutical composition comprising the 10 mg dosage form of the name-brand extended dosage form of glipizide, Glucotrol® XL, available from Pfizer, New York, N.Y. US. Referring to FIG. 4, there is shown a superimposed graph of the kinetic drug release rate of a pharmaceutical composition comprising the substances listed in Table I in the form of a perforated tablet comprising a 300 mg, 9 mm diameter tablet with a 3.2 mm perforation, and the kinetic drug release rate of Glucotrol® XL. Both pharmaceutical compositions were initially exposed to water for 2 hours at 50 rpm. As shown in FIG. 4, there was substantially no release of glipizide from the perforated tablet over the initial 2 hour time period, whereas the Glucotrol® XL tablets released glipizide upon initial exposure to water. The perforated tablet comprising glipizide and the Glucotrol® XL tablets were then placed in a pH 6.8 solution prepared from 0.01 M NaH₂PO₄ and 0.01 M Na₂HPO₄ in 0.1 M NaCl, at 50 rpm. As shown in FIG. 4, glipizide was then released from the perforated tablet at a substantially constant linear release rate. As also shown in FIG. 4, the kinetic drug release rate is increased from the kinetic drug release rate of that shown in FIG. 3 due to the wetting of the surface of the perforated tablets by placing the tablets in water prior to the pH increase.

### EXAMPLE III

### Comparative Kinetic Drug Release Rates of a Perforated Tablet and Varying Dosages of Extended Release Tablets

According to another embodiment of the present invention, a composition for a perforated tablet comprises the substances listed in Table II.

TABLE II

| | PERCENT WEIGHT |
|---|---|
| SUBSTANCE | OF TOTAL WEIGHT |
| Cellulose acetate phthalate | 9.3 |
| Ethylcellulose | 37.2 |
| Eudragit ® S | 9.3 |
| Hydroxypropylmethylcellulose acetate succinate | 9.3 |
| Hydroxypropylmethylcellulose phthalate | 9.3 |
| Kollicoat ® MAE | 9.3 |
| Methylcellulose E15 | 9.3 |
| Nifedipine | 7.0 |

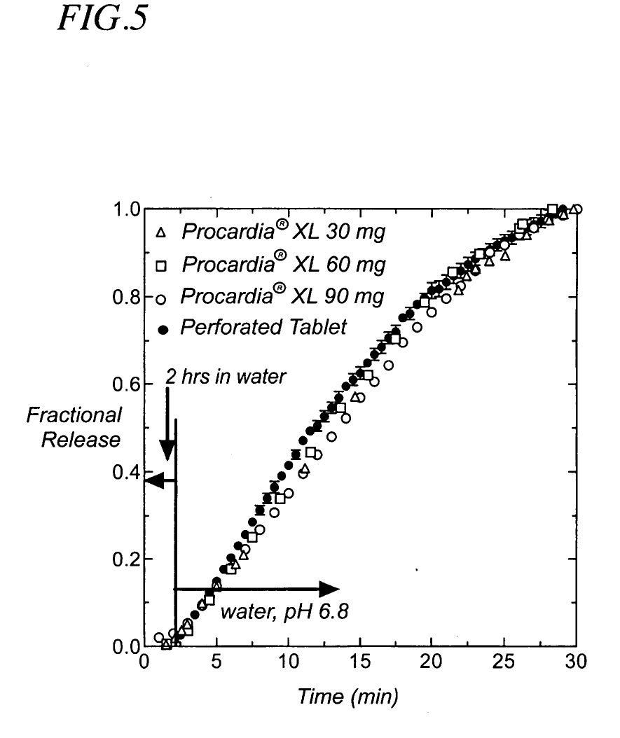

Referring to FIG. 5, there is shown a superimposed graph of the kinetic drug release rate of a pharmaceutical composition in the form of a perforated tablet and the substances listed in Table II and the kinetic drug release rate of 30 mg, 60 mg and 90 mg dosage forms of the name-brand extended dosage form of nifedipine, Procardia® XL, available from Pfizer, New York, N.Y. US. FIG. 5 shows the kinetic drug release rate of a pharmaceutical composition comprising the substances listed in Table II from a perforated tablet comprising a 10 mm diameter, 400 mg tablet with a 2.8 mm perforation, and the kinetic drug release rates of 30 mg, 60 mg, and 90 mg dosage forms of Procardia® XL tablets in a pH 6.8 solution prepared from 0.01 M NaH₂PO₄ and 0.01 M Na₂HPO₄ in 0.1 M NaCl and 0.6% Sodium Lauryl Sulfate, at 50 rpm. The perforated tablet comprising nifedipine and the 30 mg, 60 mg, and 90 mg dosage forms of Procardia® XL were exposed to water for 2 hours at 50 rpm. As shown in FIG. 4, substantially no nifedipine was released from the perforated tablet over the initial 2 hour time period, whereas each of the 30 mg, 60 mg, and 90 mg dosage forms of Procardia® XL released nifedipine upon initial exposure to water. The perforated tablet comprising nifedipine and the 30 mg, 60 mg, and 90 mg dosage forms of Procardia® XL were then placed in the pH 6.8 solution. As shown in FIG. 5, the perforated tablet then released nifedipine at a substantially constant linear release rate.

### EXAMPLE IV

### Kinetic Drug Release Rate of a Perforated Layered Tablet

According to another embodiment of the present invention, a composition for a perforated layered tablet comprises the substances listed in Table III.

TABLE III

| | PERCENT WEIGHT |
|---|---|
| | OF TOTAL WEIGHT |
| INNER LAYER SUBSTANCE | |
| ethylcellulose | 19.34 |
| Eudragit ® S | 19.34 |
| Kollicoat ® MAE | 19.34 |
| Hydroxypropylmethylcellulose acetate succinate | 19.34 |
| Methylcellulose E15 | 19.34 |
| Glipizide | 3.3 |
| OUTER LAYER SUBSTANCE | |
| ethylcellulose | 100 |

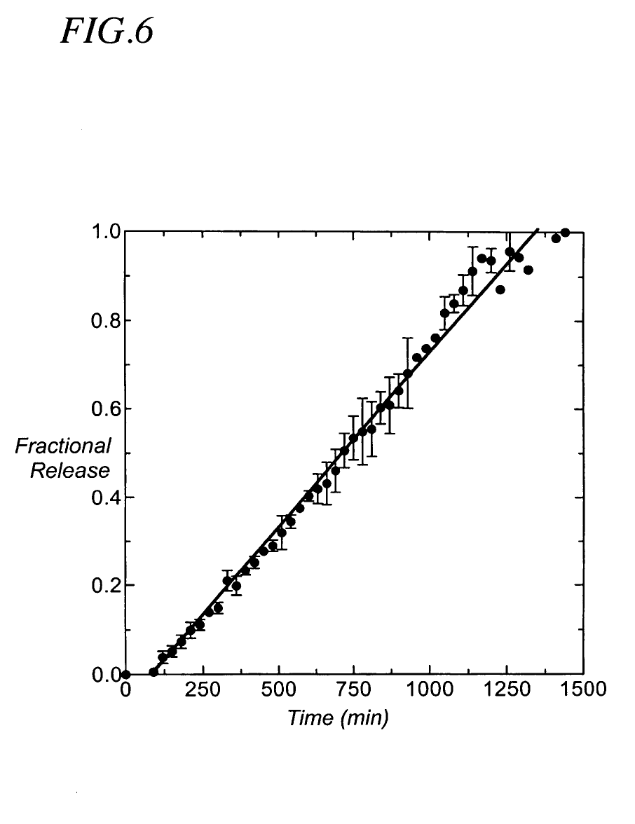

Referring to FIG. 6, there is shown a graph of the kinetic drug release rate of glipizide, from a pharmaceutical composition in the form of a perforated layered tablet and the substances listed in Table III. FIG. 6 shows the kinetic drug release rate of a pharmaceutical composition comprising the substances listed in Table III in a perforated tablet form comprising a 500 mg, 10 mm diameter tablet with a 3.2 mm perforation, in a pH 6.8 solution prepared from 0.01 M NaH₂PO₄ and 0.01 M Na₂HPO₄ in 0.1 M NaCl, at 50 rpm. The perforated layered tablet comprises a 300 mg inner layer of the inner layer substances listed in Table III, a 100 mg top outer layer and a 100 mg bottom outer layer, respectively, of the outer layer substances listed in Table III. As shown in FIG. 6, the kinetic drug release rate of glipizide from the perforated layered tablet is small even at enteric pH, such as a 6.8 pH, and the kinetic drug release rate is a substantially constant linear release rate.

In the perforated layered tablets of the present invention, the release of the drug through the lateral direction is blocked by layering the perforated tablets with a water-insoluble polymer or a water-soluble polymer. A theoretical kinetic drug release rate for the perforated layered tablet can be expressed by:

$$\frac{{M}_{t}}{{M}_{∞}} = \frac{{k}_{e}}{{C}_{o} ( {r}_{o} - {r}_{i} )} t \qquad ( 2 )$$

where Mₜ/M_{∞} is the fractional release rate, kₑ is the constant erosion rate of enteric polymers, rₒ is the radius of the tablet, rᵢ is the radius of the central hole, Cₒ is the initial drug concentration in the tablet, and t is the release time. As shown by Equation (2) the rate is independent of the thickness of the tablet. Thus, the same kinetic drug release rate in different drug loading formulations can be obtained by adding more drugs while the same composition proportions are maintained.

Although the present invention has been discussed in considerable detail with reference to certain preferred embodiments, other embodiments are possible. Therefore, the scope of the appended claims should not be limited to the description of preferred embodiments contained herein.

1. A pharmaceutical composition for the controlled release of a drug in the form of a perforated tablet, the pharmaceutical composition comprising one or more than one enteric polymer and a drug, where the enteric polymer is substantially hydrophobic and substantially soluble in a substantially aqueous environment above a pH of about 5.

2. The pharmaceutical composition according to claim 1, where the pharmaceutical composition comprises a plurality of layers, where one or more than one of the plurality of layers is a substantially water-insoluble polymer, or a substantially water-soluble polymer, and where one or more than one of the plurality of layers comprises an enteric polymer and a drug.

3. The pharmaceutical composition according to claim 1, where the form of the perforated tablet is a cylindrically shaped tablet, and where the perforation extends completely through the center of the cylindrically shaped tablet.

4. The pharmaceutical composition according to claim 1, where the one or more than one enteric polymer is selected from the group consisting of a hydroxypropylmethylcellulose acetate succinate, a hydroxypropylmethylcellulose phthalate, a polyvinylacetate, and a polyacrylate.

5. The pharmaceutical composition according to claim 3, where the one or more than one enteric polymer is a polyacrylate selected from the group consisting of an acrylate polymer, a methacrylate polymer, a methylmethacrylate polymer, an ethylacrylate polymer, a copolymer comprising a combination of the preceding polymers, and a carboxylic acid functional group containing derivative of the preceding polymers and copolymers.

6. The pharmaceutical composition according to claim 4, where the one or more than one enteric polymer is a polyacrylate selected from the group consisting of a methacrylic acid-methylmethacrylate copolymer and a methacrylic acid-ethylacrylate copolymer.

7. The pharmaceutical composition according to claim 1, where the one or more than one enteric polymer is present in an amount effective to control the release of the drug at a substantially constant linear rate over time, or a slightly increasing linear rate over time, or a slightly decreasing linear rate over time.

8. The pharmaceutical composition according to claim 1, where the enteric polymer is present in an amount of between about 1% and about 99%.

9. The pharmaceutical composition according to claim 1, where the enteric polymer is present in an amount of between about 20% and about 75%.

10. The pharmaceutical composition according to claim 1, where the enteric polymer is present in an amount of between about 35% and about 65%.

11. The pharmaceutical composition according to claim 1, additionally comprising one or more than one binder.

12. The pharmaceutical composition according to claim 11, where the binder is selected from the group consisting of a water-soluble cellulose, a polyethylene oxide, a polyethylene glycol, a water-insoluble cellulose, a water-insoluble polyvinylacetate, and a water-insoluble polyacrylate.

13. The pharmaceutical composition according to claim 12, where the one or more than one binder is a water-insoluble polyacrylate selected from the group consisting of a water-insoluble acrylate polymer, a water-insoluble methacrylate polymer, a water-insoluble methylmethacrylate polymer, a water-insoluble ethylacrylate polymer, a copolymer comprising a combination of the preceding polymers, a water-insoluble quaternary ammonium functional group containing derivative of the preceding polymers and copolymers, and a water-insoluble ester functional group containing derivative of the preceding polymers and copolymers.

14. The pharmaceutical composition according to claim 13, where the one or more than one binder is a water-insoluble polyacrylate selected from the group consisting of an acrylate-methacrylate copolymer with a quaternary ammonium functional group, an ethylacrylate-methylmethacrylate copolymer with a neutral ester functional group, and an (ethylacrylate, methylmethacrylate) polymer dispersion.

15. A method of making a pharmaceutical composition for the controlled release of a drug in the form of a perforated tablet, the method comprising:
a) mixing one or more than one enteric polymer and a drug to form a mixture, where the enteric polymer is substantially hydrophobic and substantially soluble in a substantially aqueous environment above a pH of about 5;
b) compressing the mixture into a tablet; and
c) forming a perforation in the tablet.

16. The method according to claim 15, additionally comprising mixing one or more than one binder into the one or more than one enteric polymer and a drug to form the mixture.

17. A pharmaceutical composition for the controlled release of a drug in the form of a perforated tablet made according to the method of claim 15.

18. The method of making the pharmaceutical composition according to claim 1, the method comprising:
a) mixing the one or more than one enteric polymer and a drug to form a mixture;
b) compressing the mixture into a tablet; and
c) forming a perforation in the tablet.

19. A pharmaceutical composition for the controlled release of a drug in the form of a perforated tablet, the pharmaceutical composition comprising:
a) one or more than one outer layer comprising one or more than one substantially water-insoluble polymer, or one or more than one substantially water-soluble polymer; and
b) an inner layer comprising one or more than one enteric polymer and a drug;
where the enteric polymer is substantially hydrophobic and substantially soluble in an aqueous environment above a pH of about 5.

20. The pharmaceutical composition according to claim 19, where the form of the perforated tablet is a cylindrically shaped tablet, and where the perforation extends completely through the center of the cylindrically shaped tablet.

21. The pharmaceutical composition according to claim 19, where the one or more than one enteric polymer is selected from the group consisting of a hydroxypropylmethylcellulose acetate succinate, a hydroxypropylmethylcellulose phthalate, a polyvinylacetate, and a polyacrylate.

22. The pharmaceutical composition according to claim 21, where the one or more than one enteric polymer is a polyacrylate selected from the group consisting of an acrylate polymer, a methacrylate polymer, a methylmethacrylate polymer, an ethylacrylate polymer, a copolymer comprising a combination of the preceding polymers, and a carboxylic acid functional group containing derivative of the preceding polymers and copolymers.

23. The pharmaceutical composition according to claim 22, where the one or more than one enteric polymer is a polyacrylate selected from the group consisting of a methacrylic acid-methylmethacrylate copolymer and a methacrylic acid-ethylacrylate copolymer.

24. The pharmaceutical composition according to claim 19, where the one or more than one enteric polymer is present in an amount effective to control the release of the drug at a substantially constant linear rate over time, or a slightly increasing linear rate over time, or a slightly decreasing linear rate over time.

25. The pharmaceutical composition according to claim 19, where the enteric polymer is present in an amount of between about 1% and about 99%.

26. The pharmaceutical composition according to claim 19, where the enteric polymer is present in an amount of between about 20% and about 75%.

27. The pharmaceutical composition according to claim 19, where the enteric polymer is present in an amount of between about 35% and about 65%.

28. The pharmaceutical composition according to claim 19, where the inner layer further comprises one or more than one binder.

29. The pharmaceutical composition according to claim 28, where the binder is selected from the group consisting of a water-soluble cellulose, a polyethylene oxide, a polyethylene glycol, a water-insoluble cellulose, and a water-insoluble polyvinylacetate, a water-insoluble polyacrylate.

30. The pharmaceutical composition according to claim 29, where the one or more than one binder is a water-insoluble polyacrylate selected from the group consisting of a water-insoluble acrylate polymer, a water-insoluble methacrylate polymer, a water-insoluble methylmethacrylate polymer, a water-insoluble ethylacrylate polymer, a copolymer comprising a combination of the preceding polymers, a water-insoluble quaternary ammonium functional group containing derivative of the preceding polymers and copolymers, and a water-insoluble ester functional group containing derivative of the preceding polymers and copolymers.

31. The pharmaceutical composition according to claim 30, where the one or more than one binder is a water-insoluble polyacrylate selected from the group consisting of an acrylate-methacrylate copolymer with a quaternary ammonium functional group, an ethylacrylate-methylmethacrylate copolymer with a neutral ester functional group, and an (ethylacrylate, methylmethacrylate) polymer dispersion.

32. The pharmaceutical composition according to claim 19, where the outer layer comprises one or more than one substantially water-insoluble polymer selected from the group consisting of a water-insoluble ethylcellulose, a water-insoluble cellulose ester, a water-insoluble polyvinylacetate, and a water-insoluble polyacrylate.

33. The pharmaceutical composition according to claim 32, where the outer layer comprises one or more than one water-insoluble polyacrylate selected from the group consisting of a water-insoluble acrylate polymer, a water-insoluble methacrylate polymer, a water-insoluble methylmethacrylate polymer, a water-insoluble ethylacrylate polymer, copolymers comprising a combination of the preceding polymers, a water-insoluble quaternary ammonium functional group containing derivative of the preceding polymers and copolymers, and a water-insoluble ester functional group containing derivative of the preceding polymers and copolymers.

34. The pharmaceutical composition according to claim 33, where the outer layer comprises one or more than one water-insoluble polyacrylate selected from the group consisting of an acrylate-methacrylate copolymer with a quaternary ammonium functional group, an ethylacrylate-methylmethacrylate copolymer with a neutral ester functional group, and an (ethylacrylate, methylmethacrylate) polymer dispersion.

35. The pharmaceutical composition according to claim 19, where the outer layer comprises one or more than one substantially water-soluble polymer selected from the group consisting of a water-soluble cellulose, a water-soluble polyethylene oxide, and a polysaccharide.

36. A method of making a pharmaceutical composition for the controlled release of a drug in the form of a perforated tablet comprising one or more than one outer layer comprising one or more than one substantially water-insoluble polymer, or one or more than one substantially water-soluble polymer; and an inner layer comprising one or more than one enteric polymer and a drug, where the enteric polymer is hydrophobic and substantially soluble in an aqueous environment above a pH of about 5, the method comprising
a) compressing a first outer layer into a die;
b) mixing an inner layer comprising one or more than one enteric polymer and a drug to form an inner layer mixture;
c) compressing the inner layer mixture into the first outer layer;
d) compressing a second outer layer into the inner layer mixture to form a tablet; and
e) forming a perforation in the tablet.

37. The method according to claim 36, additionally comprising mixing one or more than one binder into the mixture comprising the one or more than one enteric polymer and a drug to form the mixture.

38. A pharmaceutical composition for the controlled release of a drug in the form of a perforated tablet made according to the method of claim 36.

39. The method of making the pharmaceutical composition according to claim 19, the method comprising:
a) compressing the first outer polymer layer into a die;
b) mixing the inner layer comprising the one or more than one enteric polymer and the drug into an inner layer mixture;
c) compressing the inner layer mixture into the first outer layer;
d) compressing the second outer polymer layer into the inner layer mixture to form a tablet; and
e) forming a perforation in the tablet.
